(19) United States
(12) Patent Application Publication
(10) Pub. No.: US 2022/0364128 A1
(43) Pub. Date: Nov. 17, 2022

(54) METHODS OF PREPARING BIOSURFACTANTS USING CARBON DIOXIDE AND/OR LIGNOCELLULOSE AS SUBSTRATE

(71) Applicant: Mikros Biochem, Costa Mesa, CA (US)

(72) Inventor: Wenjie Xia, Irvine, CA (US)

(73) Assignee: Mikros Biochem, Costa Mesa, CA (US)

(21) Appl. No.: 17/743,145

(22) Filed: May 12, 2022

Related U.S. Application Data
(60) Provisional application No. 63/187,890, filed on May 12, 2021.

(30) Foreign Application Priority Data
Jun. 8, 2021 (CN) 2021-10639723.6

(51) Int. Cl. C12P 7/10; C12P 21/00; C12P 19/00; C12N 1/20

(52) U.S. Cl. CPC C12P 7/10; C12P 21/00; C12P 19/00; C12N 1/20; C12P 2201/00; C12P 2203/00

(57) ABSTRACT

Unique carbon dioxide or lignocellulosic substrate is prepared and used to produce biosurfactants, based on different types of microorganism fermenting strains, using carbon dioxide or lignocellulose-based raw materials as the primary feedstock, subsequently utilizing a fermentation process to synthesize different structures of biosurfactants. This is a two-phase reaction where phase-one creates the feedstock for the phase-two reactions. The fermentation broth resulting from the phase-two reaction is the crude biosurfactant; it uses glycolipid or lipopeptide biosurfactant as the main component. The broth is then refined by filtration, then concentrated, and further purified to obtain the pure biosurfactant material. The biosurfactant of the present disclosure can be applied to industries such as petroleum, food or agriculture, daily chemicals, industrial chemicals, environmental protection, and medicine.

### CROSS-REFERENCE TO RELATED APPLICATION

This application claims priority to and the benefit of the earlier filing of U.S. Provisional Application No. 63/187,890, filed on Apr. 12, 2021, and claims priority to China Application No. 2021-10639723.6, filed Jun. 8, 2021; both of which are incorporated by reference herein in their entirety.

### FIELD OF THE DISCLOSURE

The disclosure relates to a preparation method of a biosurfactant using carbon dioxide or lignocellulose as a substrate, which can not only reduce carbon emission, but also synthesize important raw materials, for instance for production of biosurfactant chemistry.

### BACKGROUND OF THE DISCLOSURE

Biosurfactants are materials produced through microorganism metabolism itself, and the material is formed with a surface-active substance, the type of substance having a particular hydrophilic group and a hydrophobic group, having a ratio of more complex chemical surfactant structure. Therefore, it can be widely used in industries such as chemical industry, medicine, cosmetics, late or enhanced oil recovery, stimulation chemistry for the oil and gas industry, and environmental pollution control. According to the structural characteristics of the biosurfactants they can be divided into polymers, lipopeptides, neutral lipids, phospholipids and so on. Currently biological surfactant producing bacteria includes Enterococcus, Pseudomonas bacteria, Streptomyces, Bacillus licheniformis, Acinetobacter, yeast and the like. These microorganisms will secrete surfactant-containing metabolites in vitro during the growth process under certain specific culture conditions, such as suitable carbon source, nitrogen source, organic nutrients, pH value and temperature. This secreted surfactant-containing metabolite (or mix of metabolites) is the biological surface-active agent. It is estimated that the cost of biosurfactants, when at commercial scale, is only 30% of the cost of synthetic surfactants. Since biosurfactants are non-toxic, from an ecological point of view, biosurfactants are more conducive to environmental protection and sustainability than synthetic surfactants. Because biosurfactants have the above advantages and can be fermented and produced through biological metabolism and other means, they have received general attention from the bioengineering community. At the same time, biosurfactants have biological activities such as anti-tumor, anti-bacterial, anti-fungal, anti-viral and anti-inflammatory.

The production cost of biosurfactants is mainly dependent on three factors: raw material cost, energy consumption, and purification cost. Of these, the raw material costs and the total cost of the purification costs are in the range of 70-80% of the total cost. Therefore, choosing low cost raw materials and improving the extraction process are key to reducing total overall product costs. Generally, the purification process and cost are impacted by the condition or purity of the raw materials. The biosurfactants that have been previously recognized in research, or commercially, are synthesized by fermentation using vegetable oil or glucose as a carbon source. Among them, vegetable oil is commonly used as it is available in a liquid state and is easily solubilized and emulsified during the fermentation process. However, this also increases the difficulty of oil-water separation and leads to an increased purification cost. At the same time, the cost of vegetable oil and glucose as substrate is relatively high when compared to other alternatives. Under the current world development trend of low-carbon emission reduction, the ability to use carbon dioxide and biomass is a popular topic however it has been recognized for technical difficulty in various studies around the world.

### DETAILED DESCRIPTION

A first object of the disclosure is to provide a production process which is simple, low cost, and makes use of carbon dioxide or lignocellulosic substrates as methods for preparing biologically produced material to be used as the feedstock of the second reaction. A second objective makes use of a multiple bacteria collaboration, symbiotic culture for production of biosurfactants. The biosurfactants involved in the present disclosure include glycolipids, fats, lipoproteins, lipopeptides, etc. Specific examples include Rhamnolipid, Sophorolipid, Surfactin, Cellulose lipid, and so forth.

In the present disclosure, biological surface-active compound (e.g., biosurfactant) preparations are produced using a multistage, multi-strain fermentation process wherein the main fermentation feedstock is carbon dioxide or lignocellulosic; biosurfactant to the fermentation broth as the main component, the biological surfactants can be glycolipids or lipopeptides. The concept of the consolidate bioprocess is employed in provided embodiments. Representative embodiments include the following:

(1) Fermentation A (a first fermentation starting culture broth): Medium for fermentation in which carbon dioxide serves as the main carbon source is inoculated with 1%-10% of microbial seed liquid, then incubated with aeration of 0.1-0.4 vvm, under light or no light conditions. Temperature is maintained at 20-40° C., and agitation intensity of 100-300 rpm. Representative medium contains: 0.1-0.5% potassium nitrate, 0.01-0.05% potassium dihydrogen phosphate, 0.01-0.2% magnesium sulphate heptahydrate, 0.01-0.05% calcium chloride dihydrate, 0.01-0.05% ferrous sulfate heptahydrate, 0.001-0.01% zinc sulfate heptahydrate, 0.001-0.01% manganese chloride tetrahydrate, 0.001-0.01% copper sulfate pentahydrate, 0.001-0.01% disodium edetate, and 0.1-0.3% calcium carbonate. Culturing is carried out for 4-10 days to produce fermentation broth A.

Microbial strains appropriate for this fermentation process include strains able to obtain their predominant carbon requirement from carbon dioxide (CO₂); these microorganisms can convert carbon dioxide to lipids, proteins, polysaccharides, fatty acids, alcohols or the like. Example fermentation microbes include, but are not limited to, species of one or more of Pyrococcus, Pseudomonas, Metallococcus sp., Metallosphaera, Rhodospirillum, Chloroflexus, Aspergillis, Cyanobacteria, Chlorella, Dunaliella, Nannochloropsis sp (Nannochloropsis), Scenedesmus (e.g., Scenedesmus obliquus), Botryococcus, and the like. Specific examples of fermentation microbes, and growth conditions, are provided herein.

(2) Fermentation B (a second fermentation starting culture broth): Medium for fermentation in which lignocellulosic material serves as the main carbon source is inoculated with 1-10% of microbial seed liquid (such as 5% seed liquid containing Trichoderma reesei (T. reesei)), then incubated at a temperature maintained at 20-40° C., with agitation intensity of 100-300 rpm. The medium is inorganic salt medium, initial pH of 7-8.5; the lignocellulosic material is added at 2-10% volume; culture medium volume was 50% (bulk volume ratio). The base medium contains: (NH₄)₂SO₄ 0.1-0.5%, MgSO₄. 7H₂O 0.01-0.05%, CuSO₄.5H₂O 0.01-0.03%, MnSO₄0.001-0.005%, and 0.1-0.5% calcium carbonate. Culturing is carried out for 4-10 days to produce fermentation broth B.

Microbial strains appropriate for this fermentation process include strains able to obtain their predominant carbon requirement from lignocellulosic material. Generally speaking, these microorganisms can grow at 20-40° C. and degrade lignocellulose into oligosaccharides, sugars, and/or small molecule acid alcohols. Representative microbial strains appropriate for this fermentation process include but are not limited to, Aurantiacus, Sordaria, Pseudomonas, Trametes, Irpex, Lenzite, Phanerochaete (e.g., white rot fungi), grams Klebsiella (Klebsiella), Ochrobactrum, Sphingobacterium, Dysgonomonas, Sphingobacterium, Bacteroides, Parabacteroides, Flavobacterium, polymorphonuclear Aeromonas, Pleomorphomonas, Arcticibacter, Elizabethkingia, Neisseria, Mycobacterium, Trichoderma (such as Trichoderma reesei), Zymomonas, Stenotrophomonas, Paenibacillus, Nocardia, Nocardiopsis, Nocardia, Bacillus, Rhizobium, Cellulomonas, Vibrio, Cellvibrio, Cytophaga, various kinds of bacilli (Alistipes), Aspergillus (such as Aspergillus flavus), Ruminofilibacter, Clostridium, and the like.

(3) Pretreatment of fermentation broth. After fermentation broths A and B are obtained, there are two ways to employ them for the subsequent synthesis of biosurfactant(s). In a first embodiment, fermentation broths A and B are directly mixed, and after mixing, they are sterilized using high temperature and the resultant product directly enters the next (secondary fermentation) step. In a second embodiment, one or both of fermentation broths A and B are respectively concentrated (that is, at least some portion of the water is removed), then the (concentrated) broths are mixed and sterilized, with the concentrated and sterilized product then used in the next process step (secondary fermentation, also referred to as Fermentation C). Whichever pretreatment method is used, it does not affect the subsequent preparation process.

(4) Fermentation C (production of biosurfactants using earlier fermentation product(s) as starting material). 5%-10% of microbial seed liquid (starter culture) is inoculated to an inorganic salt culture medium, wherein the mixed fermentation microbial biomass and fermentation products produced in earlier step(s) provides the carbon source for this secondary fermentation. The fermentation temperature is 20-40° C., with agitation at 100-300 rpm, and ventilation of 0.1-0.4 vvm. The fermentation culturing is carried out for 3-10 days to obtain fermentation broth C. Other than the carbon source (produced in stages 1-3, above), this fermentation culture medium includes components: NaNO₃: 0.1-1.4%, FeCl₂: 0.002-0.006%, NaH₂PO₄: 0.25-1.5%, K₂HPO₄ 0.25-1.8%, MgSO₄.7H₂O: 0.005-0.015%, KC1: 0.05%-0.3%; Choline chloride: 0.05%-0.3%; yeast extract: 0.001 to 0.1%; and trace elements: Zn, Mn, Calif. The pH of the medium at inoculation is pH 6-7.

Microbial strains appropriate for this fermentation process include strains able to obtain their predominant carbon requirement from the biomass and biological products that are produced in Fermentation A, Fermentation B, or both, and which synthesize (e.g., as an anabolite) at least one biosurfactant compound. Appropriate microorganisms grow at 20-40° C. and biosynthesize glycolipids and/or lipopeptides from sugar, alcohol, acids, fat, protein, and other cellular substances. Microbial strains appropriate for this fermentation process include biosurfactant synthesis fermenting microbes, such Pseudomonas, Bacillus, Candida, Acinetobacter, Pantoea, Sphingomonas, Zymomonas, Streptomyces, Rhodococcus, Pseudozyma, Ustilaginales (e.g., smut fungus), Moesziomyces (mohs Ustilago) and the like.

(5) Separation, purification and concentration: Fermentation broth C, obtained as escribed above, is sterilized (for instance, by heating at 80-120° C. for 2 hours), the pH is adjusted to pH=8.0-10.0, and the resulting mix is subjected to three-phase high-speed centrifugation (*10000 g), to remove the microbial solids. Avoiding the microbial solids and residual oil (which floats), the clear liquid in the middle layer is harvested. The pH of the clear liquid in the middle layer is adjusted to pH 2-3 (for acid precipitation), and refrigerated for 24 hours at a temperature of 4-10° C. The precipitate is collected (for instance, by filtration involving passing the chilled and acidified liquid through a ceramic membrane at 4-10° C.). The captured precipitate is dissolved on an aqueous solution with a pH of >8, then filtered again (for instance, by passage through a ceramic membrane) to obtain an aqueous solution of biological surface active (biosurfactant) molecules. Optionally, the biosurfactant preparation can be concentrated (e.g., 10-15 fold) under vacuum at 50° C.

(6) Structure identification: Optionally, the structure of produced biosurfactant(s) can be analyzed in order to identify one or more specific components. By way of example, HPLC-MS or the like may be used to identify biosurfactant structure(s) as a glycolipid, or a lipopeptide, or a mixture of both thereof.

Biosurfactants produced by the methods provided herein can be used in any situation in which a biosurfactant is useful. This includes as an ingredient in a mixed composition in various applications, including shampoo, cleaner, pesticide, lubricants, agricultural compositions, etc.

### BRIEF DESCRIPTION OF THE DRAWING

FIG. 1 shows the structure of representative biosurfactants.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A multi-stage, multi-strain fermentation method, including: a first fermentation process, including contacting one or more strains of microorganism(s) with a primary feed stock including carbon dioxide, which first fermentation process produces a first fermentation broth; a second fermentation process, including contacting one or more strains of microorganism(s) with a primary feed stock including lignocellulosic material, which second fermentation process produces a second fermentation broth; and a third fermentation process, including contacting a mixture of the first fermentation broth and the second fermentation broth with one or more strains of microorganism under conditions and for a time sufficient to produce a biosurfactant fermentation broth, which biosurfactant fermentation broth includes at least one biosurfactant compound.

2. The fermentation method of embodiment 1, wherein the at least one biosurfactant compound includes a glycolipid or a lipopeptide.

3. The method of embodiment 1, which converts approximately one ton of sequestered CO₂ into 0.5 to 4.0 tons of biosurfactant.

4. The method of embodiment 1, wherein the one or more strains of microorganism(s) produce one or more lignocellulose decomposing enzymes, which enzymes convert lignocellulose into oligo-saccharide(s), monosaccharide(s), and/or small molecule acid species.

5. The method of embodiment 4, wherein the oligo-saccharide(s), monosaccharide(s), and/or small molecule acid species, and/or cells of the photo-autotrophic or chemo-autotrophic microorganism(s), are substrate(s) for the second fermentation process.

6. The method of embodiment 1, wherein the biosurfactant fermentation broth is further processed to extract biosurfactant.

7. The method of embodiment 1, further including a biosurfactant extraction process, the biosurfactant extraction process including: fractionating the biosurfactant fermentation broth to produce an aqueous phase; acid precipitating the aqueous liquid; filtering the acid precipitated aqueous liquid to obtain the precipitate; dissolving the precipitate with an aqueous liquid at pH>8 to produce a resuspended precipitate; filtering the resuspended precipitate to obtain a aqueous solution containing the biosurfactant.

8. The method of embodiment 7, wherein the biosurfactant fermentation broth fractionation includes adjusting the biosurfactant fermentation broth to pH=8.0-10.0 to produce a pH-adjusted broth, centrifuging the pH-adjusted broth at high speed (10000×g) to produce a microbial material pellet, a relatively clear aqueous liquid layer, and a lipid phase.

9. The method of embodiment 7, wherein the acid precipitation includes adjusting the relatively clear aqueous liquid to pH=2-3 to produce an acidic aqueous liquid, then chilling the acidic aqueous liquid at 4-10° C. for 24 hours.

10. The method of embodiment 7, wherein filtering the acid precipitated aqueous liquid includes filtering through a 0.1 μm ceramic membrane at 4-10° C.

11. The method of embodiment 7, wherein filtering the resuspended precipitate includes filtering through a 0.1 μm ceramic membrane.

12. The method of embodiment 7, further including concentrating the biological surface-active aqueous solution under vacuum at 50° C.

13. The method of embodiment 1, wherein at least one microorganism of the first fermentation is selected from the following: Pyrococcus, Pseudomonas, Metallococcus sp., Metallosphaera, Rhodospirillum, Chloroflexus, Aspergillis, Cyanobacteria, Chlorella, Dunaliella, Nannochloropsis, Scenedesmus, and Botryococcus.

14. The method of embodiment 1, wherein at least one microorganism of the second fermentation is selected from the following: Aurantiacus, Sordaria, Pseudomonas, Trametes, Irpex, Lenzite, Phanerochaete, grams Klebsiella (Klebsiella), Ochrobactrum, Sphingobacterium, Dysgonomonas, Sphingobacterium, Bacteroides, Parabacteroides, Flavobacterium, polymorphonuclear Aeromonas, Pleomorphomonas, Arcticibacter, Elizabethkingia, Neisseria, Mycobacterium, Trichoderma (such as Trichoderma reesei), Zymomonas, Stenotrophomonas, Paenibacillus, Nocardia, Nocardiopsis, Nocardia, Bacillus, Rhizobium, Cellulomonas, Vibrio, Cellvibrio, Cytophaga, Alistipes, Aspergillus (such as Aspergillus flavus), Ruminofilibacter, and Clostridium.

15. The method of embodiment 1, wherein at least one microorganism of the third fermentation is selected from the following: Pseudomonas, Bacillus, Candida, Acinetobacter, Pantoea, Sphingomonas, Zymomonas, Streptomyces, Rhodococcus, Pseudozyma, Ustilaginales, and Moesziomyces.

16. The method embodiment 1, wherein the first fermentation process and the second fermentation process: are carried out at least partially in separate containers; are carried out at least partially in the same container; or are substantially carried out in the same container (that is, mixed).

17. The method of embodiment 1, wherein the lignocellulose is from one or more of corn stalks, straws, leaves, and wood chips.

18. The method of embodiment 1, wherein the first fermentation process is carried out in a first culture condition including: culturing the microorganism(s) using mineral medium, carbon dioxide as the main carbon source, aeration of 0.01-0.4 vvm, and either light or no light conditions.

19. The method of embodiment 17, wherein the first culture condition further includes an initial inoculum of 1-10% stock culture, temperature control 20-40° C., and agitation intensity 100-300 rpm.

20. The method of embodiment 17, wherein the first culture condition include a medium including: potassium nitrate of 0.1-0.5%, potassium dihydrogen phosphate of 0.1-0.5%, magnesium sulphate heptahydrate of 0.01-0.2%, calcium chloride dihydrate of 0.01-0.05%, ferrous sulfate heptahydrate of 0.01-0.05%, zinc sulfate heptahydrate of 0.001-0.01%, manganese chloride tetrahydrate of 0.001-0.01%, copper sulfate pentahydrate of 0.001-0.01%, disodium edetate of 0.001-0.01%, and calcium carbonate of 0.1-0.3%.

21. The method of embodiment 1, wherein the first fermentation process is carried out for 4-10 days.

22. The method of embodiment 1, wherein the microorganism(s) of the second fermentation are cultured using mineral medium, with 2-10% lignocellulose as the main carbon source, maintained at 20-40° C., with stirring at 100-300 rpm, and an initial medium pH of 7-8.5.

23. The method of embodiment 22, wherein the culture medium volume was 50% (by volume); 10% inoculum (from seed stock).

24. The method of embodiment 22, wherein the medium includes: (NH₄)₂SO₄ 0.1-0.5%, MgSO_{4 .}7H₂O 0.01-0.05%, CuSO_{4.} 5H₂O 0.01-0.03%, MnSO₄ 0.001-0.005%, and calcium carbonate 0.1-0.5%.

25. The method of embodiment 22, wherein the first and/or second fermentation process is carried out for 4-10 days.

26. The method of embodiment 1, wherein the third fermentation process is carried out using an inorganic salt medium, wherein the carbon source is a mixture of the first fermentation broth and the second fermentation broth.

27. The method of embodiment 26, wherein the mixture of the first and second fermentation broths is included in the third fermentation: at a dosage of 5-15 wt %; or at a dosage of 2-10 wt %.

28. The method of embodiment 26, wherein the temperature of the third fermentation is 20-40° C., the agitation intensity is 100-300 rpm, and ventilation is at a rate of 0.1-0.4 vvm.

29. The method of embodiment 26, wherein the third fermentation process is carried out for 3-10 days.

30. The method of embodiment 26, wherein the culture medium is at pH 6-7 and includes: NaNO₃: 0.1-1.4%, FeCl_{2: 0.002}-0.006%, NaH₂PO₄: 0.25-1.5%, K₂HPO₄: 0.25-1.8%, MgSO6hd 4.7H₂O: 0.005-0.015%, KCl: 0.05%-0.3%; Choline chloride: 0.05%-0.3%; yeast extract: 0.001-0.1%, and trace elements: Zn, Mn, Ca.

31. A biological surface-active agent (biosurfactant) produced using the method of embodiment 1.

### EXAMPLES

### Example 1

### Representative Surface-Active Biological Preparation Method

This Example describes a first specific embodiment of production of biosurfactants using the general methods provided herein.

(1) Fermentation A: 2% of Scenedesmus obliquus (Scenedesmus) seed liquid culture was inoculated into first fermentation medium prepared with carbon dioxide as the main carbon source. Fermentation took place with aeration of 0.01 vvm, under illumination, temperature controlled to 25° C., and agitation intensity 100 rpm. The fermentation was continued 7 days, to produce fermentation broth A.

The first fermentation medium contained:

| | |
|---|---|
| potassium nitrate | 0.15% |
| potassium dihydrogen phosphate | 0.1% |
| magnesium sulphate heptahydrate | 0.01% |
| calcium chloride dihydrate | 0.01% |
| ferrous sulfate heptahydrate | 0.01% |
| seven hydrated zinc sulfate | 0.001% |
| manganese chloride tetrahydrate | 0.002% |
| copper sulfate pentahydrate | 0.001% |
| disodium edetate | 0.001% |
| calcium carbonate | 0.1% |

(2) Fermentation B: 5%-10% of T. reesei (Trichoderma reesei) seed liquid culture was inoculated into second fermentation medium (initial pH 7-8.5), which was inorganic salt medium containing corn stalks as the main carbon source. The fermentation took place at controlled temperature 30° C., with agitation intensity 300 rpm. Cornstalk material was 5%; culture medium liquid volume was 50% (volume ratio). The fermentation was continued for 10 days, to produce fermentation broth B.

The second fermentation medium contained:

| | |
|---|---|
| (NH₄)₂SO₄ | 0.1% |
| MgSO₄•7H₂O | 0.01% |
| CuSO₄•5H₂O | 0.01% |
| MnSO₄ | 0.001% |
| calcium carbonate | 0.1% |

(3) Concentrate: Fermentation broths A and B were concentrated by 50% respectively, mixed, then sterilized before they were used directly in the next step.

(4) Fermentation C. 10% of Pseudomonas seed liquid culture was inoculated into third fermentation medium, which was inorganic salt medium wherein the carbon source was a mixture of prepared fermentation broth A concentrate (8% of the weight of the culture medium) and prepared fermentation broth B concentrate (2% of the weight of the culture). Fermentation C was carried out at 33° C., stirring intensity 200 rpm, ventilation of 0.3 vvm. The fermentation was continued for 5 days to obtain fermentation broth C.

(5) Separation, purification and concentration: Fermentation broth C obtained from the above fermentation process was sterilized at 120° C. for 2 hours, adjusted to pH=8.0-10.0, and subjected to three-phase high-speed centrifugation (10000 ×g) to remove the bacteria and residual slag. The middle fraction was a clear solution. The pH of the clear liquid in the middle layer was adjusted to 2-3, then refrigerated for 24 hours at a temperature of 4-10° C. The resulting precipitate was collected through a ceramic membrane (0.1 μm) at 4-10° C., and dissolved with an aqueous solution with a pH of >8. The solution was then passed through ceramic membrane to obtain No. 1 biosurfactant. Finally, this initial preparation was concentrated 10-15 fold at 50° C. under vacuum.

(6) Structure identification: The structure was identified as a glycolipid type biosurfactant. See Example 7.

### Example 2

### A Method for Preparing a Biological Surface Activity Composition

This Example describes a second specific embodiment of production of biosurfactants using the general methods provided herein.

(1) Fermentation A: 5% of Dunaliella seed liquid culture was inoculated into third fermentation medium prepared with carbon dioxide as the main carbon source. Fermentation took place with aeration of 0.01 vvm, under illumination, temperature controlled to 23° C., and agitation/stirring intensity 200 rpm. The fermentation was continued 7 days, to produce fermentation broth A.

The first fermentation medium contained:

| | |
|---|---|
| potassium nitrate | 0.3% |
| potassium dihydrogen phosphate | 0.16% |
| magnesium sulphate heptahydrate | 0.03% |
| calcium chloride dihydrate | 0.021% |
| ferrous sulfate heptahydrate | 0.016% |
| zinc sulfate heptahydrate | 0.002% |
| manganese chloride tetrahydrate | 0.0027% |
| copper sulfate pentahydrate | 0.003% |
| disodium edetate | 0.003% |
| calcium carbonate | 0.2% |

(2) Fermentation B: 10% of the Lenzites seed liquid was inoculated into second fermentation medium (initial pH 7-8.5), which is inorganic salt medium containing straw as the main carbon source. The fermentation took place at controlled temperature 27° C., with agitation/stirring intensity 200 rpm. Straw material was 6%; culture medium liquid volume was 50% (volume ratio). The fermentation was continued for 10 days, to produce fermentation broth B.

The second fermentation medium contained:

| | |
|---|---|
| (NH₄)₂SO₄ | 0.1% |
| MgSO₄•7H₂O | 0.01% |
| CuSO₄•5H₂O | 0.01% |
| MnSO₄ | 0.001% |
| calcium carbonate | 0.1% |

(3) Concentrate: Fermentation broths A and B were concentrated by 50% respectively, mixed, then sterilized before they were used directly in the next step.

(4) Fermentation C: 10% of Candida seed liquid was inoculated into third fermentation medium, which was inorganic salt medium wherein the carbon source was a mixture of prepared fermentation broth A concentrate (1% of the weight of the culture medium) and prepared fermentation broth B concentrate (10% of the weight of the culture). Fermentation C was carried out at 25° C., with stirring/agitation intensity 300 rpm, ventilation of 0.3 vvm, cultured 10 days to obtain broth C.

(5) Separation, purification and concentration: The above-described fermentation broth C was sterilized by autoclaving at 120° C. for 2 hours, then subjected to hree-phase high-speed centrifugation (10000 ×g) to remove the bacteria and residual slag. The middle fraction was a brown viscous liquid. The pH of the clear liquid in the middle layer was adjusted to 2-3, then refrigerated for 24 hours at a temperature of 4-10° C. The resulting precipitate was collected through a ceramic membrane (0.1 μm) at 4-10° C., and dissolved with an aqueous solution with a pH of >8. The solution was then passed through ceramic membrane to obtain No. 2 biosurfactant.

(6) Structure identification: The structure was identified as a glycolipid type. See Example 7.

### Example 3

### A Method for Preparing a Biological Surface Activity Composition

This Example describes a third specific embodiment of production of biosurfactants using the general methods provided herein.

(1) Fermentation A: 10% of Rhodospirillum seed liquid culture was inoculated into first fermentation medium prepared with carbon dioxide as the main carbon source. Fermentation too place with aeration of 0.03 vvm, under illumination, temperature controlled to 26° C., and agitation/stirring intensity 200 rpm. Culture 5 days to give the fermentation broth A.

The first fermentation medium contained:

| | |
|---|---|
| potassium nitrate | 0.4% |
| potassium dihydrogen phosphate | 0.26% |
| magnesium sulphate heptahydrate | 0.05% |
| calcium chloride dihydrate | 0.021% |
| ferrous sulfate heptahydrate | 0.04% |
| zinc sulfate heptahydrate | 0.005% |
| manganese chloride tetrahydrate | 0.005% |
| copper sulfate pentahydrate | 0.003% |
| disodium edetate | 0.003% |
| calcium carbonate | 0.4% |

(2) Fermentation B: 8% of Nocardiosis seed liquid was inoculated into second fermentation medium (initial pH 7-8.5), which was inorganic salt medium containing wood as the main carbon source. The fermentation took place at temperature controlled to 28° C., agitation/stirring intensity 300 rpm. Wood (in the form of sawdust) was added to 6%; culture medium liquid volume was 50% (volume ratio). The fermentation was continued for 10 days, to produce fermentation broth B.

The second fermentation medium contained:

| | |
|---|---|
| (NH₄)₂SO₄ | 0.1% |
| MgSO₄•7H₂O | 0.01% |
| CuSO₄•5H₂O | 0.01% |
| MnSO₄ | 0.001% |
| calcium carbonate | 0.1% |

(3) Concentrate: Fermentation broths A and B were concentrated by 50% respectively, mixed, and then sterilized before they were used directly in the next step.

(4) Fermentation C: 10% of Bacillus seed liquid was inoculated into third fermentation medium, which was inorganic salt medium wherein the carbon source was a mixture of prepared fermentation broth A concentrate (1% of the weight of the culture medium) and prepared fermentation broth B concentrate (1.5% of the weight of the culture). Fermentation C was carried out at 30° C., stirring intensity 300 rpm, ventilation of 0.3 vvm. The fermentation was continued for 7 days to obtain fermentation broth C.

(5) Separation, purification and concentration: Fermentation broth C obtained from the above fermentation process was sterilized at 120° C. for 2 hours, adjusted to pH=8.0-10.0, and subjected to three-phase high-speed centrifugation (10000 ×g) to remove the bacteria and residual slag. The middle fraction was a clear solution. The pH of the clear liquid in the middle layer was adjusted to 2-3, then refrigerated for 24 hours at a temperature of 4-10° C. The resulting precipitate was collected through a ceramic membrane (0.1 μm) at 4-10° C., and dissolved with an aqueous solution with a pH of >8. The solution was then passed through ceramic membrane to obtain No. 3 biosurfactant. Finally, this initial preparation was concentrated 10-15 fold at 50° C. under vacuum.

(6) Structure identification: The structure was identified as lipopeptide class. See Example 7.

### Example 4

### Another Representative Surface Active Biological Production Process

This Example describes a fourth specific embodiment of production of biosurfactants using the general methods provided herein.

(1) Fermentation A: 5% of Chlorella seed liquid culture was inoculation into first fermentation medium prepared with carbon dioxide as the main carbon source. Fermentation took place with aeration of 0.04 vvm, under illumination, temperature controlled to 26° C., and agitation intensity 300 rpm. The fermentation was continued 7 days, to produce fermentation broth A.

The first fermentation medium contained:

| | |
|---|---|
| potassium nitrate | 0.5% |
| potassium dihydrogen phosphate | 0.2% |
| magnesium sulphate heptahydrate | 0.05% |
| calcium chloride dihydrate | 0.041% |
| ferrous sulfate heptahydrate | 0.26% |
| zinc sulfate heptahydrate | 0.005% |
| manganese chloride tetrahydrate | 0.0027% |
| copper sulfate pentahydrate | 0.003% |
| disodium edetate | 0.003% |
| calcium carbonate | 0.2% |

(2) Fermentation B: 10% of Ochrobactrum seed liquid culture was inoculated into second fermentation medium (initial pH 7-8.5), which was inorganic salt medium containing straw as the main carbon source. The fermentation took place at controlled temperature 27° C., stirring intensity 200 rpm. Straw (lignocellulosic material) was added to 9%; culture medium liquid volume was 50% (volume ratio). The fermentation was continued for 10 days, to produce fermentation broth B.

The second fermentation medium contained:

| | |
|---|---|
| (NH₄)₂SO₂ | 0.3% |
| MgSO₄•7H₂O | 0.04% |
| CuSO₄ 5H₂O | 0.021% |
| MnSO₄ | 0.0021% |
| calcium carbonate | 0.13% |

(3) Concentrate: Fermentation broths A and B were concentrated by 50% respectively, mixed, and then sterilized before they were used directly in the next step.

(4) Fermentation C: 10% of Moesziomyces seed liquid culture was inoculated into third fermentation medium, which was inorganic salt medium wherein the carbon source was a mixture of prepared fermentation broth A concentrate (10% of the weight of the culture medium) and prepared fermentation broth B concentrate (10% of the weight of the culture). Fermentation C was carried out at 26° C., stirring intensity 300 rpm, aeration of 0.4 vvm. The fermentation was continued for 10 days to obtain fermentation broth C.

(5) Separation, purification and concentration: Fermentation broth C obtained from the above fermentation process was sterilized at 120° C. for 2 hours, adjusted to pH=8.0-10.0, and subjected to three-phase high-speed centrifugation (10000 ×g) to remove the bacteria and residual slag. The middle fraction was a brown viscous liquid. As above, the liquid was subject to acidic precipitation at 10° C., the precipitate was collected through a ceramic membrane, to obtain No. 4 biosurfactant.

(6) Structural identification: The structure was identified as glycolipids. See Example 7.

### Example 5

### Further Representative Surface Active Biological Production Process

This Example describes a fifth specific embodiment of production of biosurfactants using the general methods provided herein.

(1) Fermentation A: 7% of the Pyrococcus seed liquid culture was inoculated into first fermentation medium formulated with carbon dioxide as the main carbon source. Fermentation took place with aeration of 0.05 vvm, under illumination, temperature control 28° C., and agitation/stirring intensity 150 rpm. The fermentation was continued 7 days, to produce fermentation broth A.

The first fermentation medium contained:

| | |
|---|---|
| potassium nitrate | 0.5% |
| potassium dihydrogen phosphate | 0.36% |
| magnesium sulphate heptahydrate | 0.05% |
| calcium chloride dihydrate | 0.021% |
| ferrous sulfate heptahydrate | 0.04% |
| zinc sulfate heptahydrate | 0.006% |
| manganese chloride tetrahydrate | 0.005% |
| copper sulfate pentahydrate | 0.003% |
| disodium edetate | 0.003% |
| calcium carbonate | 0.4% |

(2) Fermentation B: 8% of Mycobacterium seed liquid culture was inoculated into second fermentation medium (initial pH 7-8.5), which was inorganic salt medium containing sawdust as the main carbon source. The fermentation too place at controlled temperature at 28° C., with agitation/stirring intensity 300 rpm. Sawdust material was added at 6%; culture medium liquid volume was 50% (volume ratio). The fermentation was cultured 10 days, to produce fermentation broth B.

The second fermentation medium contained:

| | |
|---|---|
| (NH₄)₂SO₄ | 0.1% |
| MgSO₄•7H₂O | 0.01% |
| CuSO₄•5H₂O | 0.01% |
| MnSO₄ | 0.001% |
| calcium carbonate | 0.1% |

(3) Concentrate: Fermentation broths A and B were concentrated by 50% respectively, mixed, and then sterilize before they were used directly in the next step.

(4) Fermentation C: 10% of Rhodococcus seed liquid culture was inoculated into third fermentation medium, which was inorganic salt medium wherein the carbon source was a mixture of prepared fermentation broth A concentrate (12% of the weight of the culture medium) and prepared fermentation broth B concentrate (2% of the weight of the culture). Fermentation C was carried out at 30° C., stirring intensity 300 rpm, aeration of 0.4 vvm. The fermentation was cultured for 7 days to obtain fermentation broth C.

(5) Separation, purification and concentration: Fermentation broth C obtained from the above fermentation process was sterilized at 120° C. autoclaved 2 hours, adjusted to pH=8.0-10.0, and subjected to three-phase high-speed centrifugation (10000 ×g) to remove the bacteria and residual slag. The middle fraction was a clear liquid, with an upper oil phase. Product was acid precipitated and cleaned as above. After filtration through the ceramic membrane, biosurfactant No. 5 was obtained. This initial preparation was concentrated 10-15 fold at 50° C. under vacuum.

(6) Structure identification: The structure was identified as a glycolipid type. See Example 7.

### Example 6

### Another Exemplar Surface-Active Biological Production Process

This Example describes a sixth specific embodiment of production of biosurfactants using the general methods provided herein.

(1) Fermentation A: 3% of Botryococcus seed liquid culture was inoculated into first fermentation medium formulated with carbon dioxide as the main carbon source. Fermentation took place with aeration of 0.04 vvm, under illumination, temperature controlled to 26° C., and stirring intensity 300 rpm. The fermentation was continued 8 days, to produce fermentation broth A.

The first fermentation medium contained:

| | |
|---|---|
| potassium nitrate | 0.45% |
| potassium dihydrogen phosphate | 0.25% |
| magnesium sulphate heptahydrate | 0.055% |
| calcium chloride dihydrate | 0.047% |
| ferrous sulfate heptahydrate | 0.026% |
| zinc sulfate heptahydrate | 0.005% |
| manganese chloride tetrahydrate | 0.0027% |
| copper sulfate pentahydrate | 0.003% |
| disodium edetate | 0.003% |
| calcium carbonate | 0.6% |

(2) Fermentation B: 6% of the A. flavus (Aspergillus) seed liquid culture was inoculated into second fermentation medium (initial pH 7-8.5) formulated as inorganic salt medium with wood (as sawdust) as the main carbon source. The fermentation too place at controlled temperature 27° C., with stirring intensity 220 rpm. Sawdust was included at 9%; culture medium liquid volume was 50% (volume ratio). The fermentation was continued for 10 days, to produce fermentation broth B.

The second fermentation medium contained:

| | |
|---|---|
| (NH₄)₂SO₄ | 0.5% |
| MgSO₄•7H₂O | 0.05% |
| CuSO₄•5H₂O | 0.025% |
| MnSO₄ | 0.0026% |
| calcium carbonate | 0.3% |

(3) Concentrate: The fermentation broths A and B concentrated by 50% respectively, mixed, and then sterilized before they were used directly in the next step.

(4) Fermentation C: The inoculated with 10% of Ustilaginales liquid seed culture was inoculated into third fermentation medium, which was inorganic salt medium wherein the carbon source was a mixture of prepared fermentation broth A concentrate (8% of the weight of the culture medium) and prepared fermentation broth B concentrate (6% of the weight of the culture). Fermentation C was carried out at 27° C., stirring intensity 300 rpm, aeration of 0.4 vvm. The fermentation was continued for 5 days to obtain fermentation broth C.

(5) Separation, purification and concentration: Fermentation broth C obtained from the above fermentation process was sterilized by autoclave at 120° C. for 2 hours later. After three-phase high-speed centrifugation (10000 ×G) carried out essential as described above, the middle layer/fraction was harvested as a brown viscous liquid. Product was acid precipitated and cleaned as above. After filtration through the ceramic membrane, biosurfactant No. 6 was obtained.

(6) Structural identification: The structure is identified as glycolipids. See Example 7.

### Example 7

### Biosurfactants Structure Determination

Glycolipid live class detection method using a surface at high temperature concentrated sulfuric acid dehydration furfural (pentose) or a furfural derivative (hexose), the agent may generate a colored compound to react with a variety of phenols, which calculate by measuring the absorbance its content, has moss phenol black-concentrated sulfuric acid method; sulfate-anthrone colorimetric method; the above methods are to be used, averaging, to eliminate errors. Glycolipid concentration was measured following the chromogenic reaction, for instance using the sulfuric acid-anthrone chromogenic method, or Sulfuric acid-orcinol chromogenic method, or by using the HPLC and UPLC-MS (Xia et al., Chem Rev. 107:2411-2502, 2007, DOI: 10.1016/j.cej.2021.128771).

The detection method of lipopeptide surfactants adopts the L-cysteine-concentrated sulfuric acid colorimetric method. The detection method of lipopeptide surfactants follows the Ninhydrin chromogenic method with the L-cysteine as standard (see, e.g., You et al., Euro J Lipid Sci Tech. 117(6):890-898, 2024, DOI: 10.1002/ejlt.201400386); or by HPLC and UPLC-MS (see, e.g., Biniarz & Lukaszewicz, App Microbiol Biotechnol. 101(11):4747-4749, 2017, doi: 10.1007/s00253-017-8272-y).

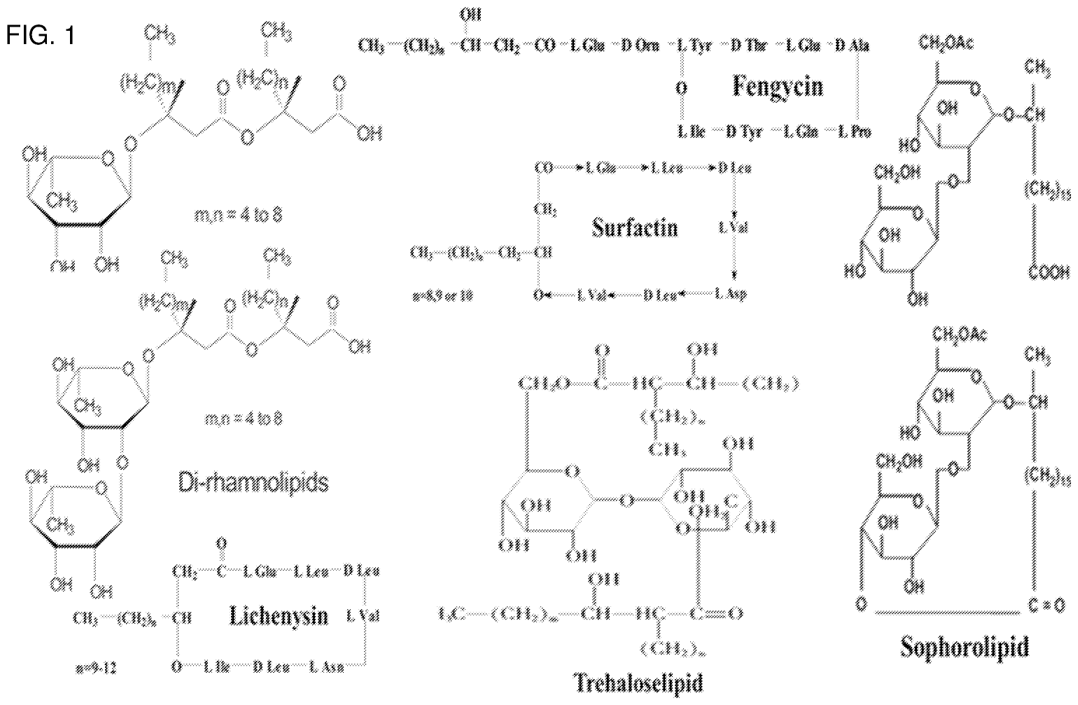

More glycolipids and lipopeptides identified specific structure like a surfactant, a corresponding standard, required by HPLC-MS or the like is determined, see Table 1 and FIG. 1.

The test results of the surfactant content in the above examples are as follows:

TABLE 1
The structure and yield of biosurfactants

| | | | Literature |
|---|---|---|---|
| | | | reported |
| Case | | Yield | evaluation |
| study | Structure | Content, g/L | output, g/L |
| Example 1 | Rhamnolipids | 46.36 | 20-30 (Ref. 1) |
| Example 2 | Sophorose | 260.93 | 90-200 (Ref. 2) |
| Example 3 | Lipopeptide | 9.36 | 0.5-2 (Ref. 3) |
| Example 4 | Mannose Erythritol | 316.39 | 100-200 (Ref. 4) |
| | Lipids (MELs) | | |
| Example 5 | Sophorose | 296.33 | 90-200 (Ref. 2) |
| Example 6 | Mannose Erythritol | 263.69 | 100-200 (Ref. 4) |
| | Lipids | | |

Table 1 Literature Key: Ref. 1: Chong & Li, Microb Cell Fact 15: 137 (12 pages), 2017 (doi.org/10.1186/s12934-017-0753-2); Ref. 2: Wongsirichot et al., J Cleaner Prod. 319: 128727, 2021 (doi.org/10.1016/j.jclepro.2021.128727); Ref. 3: Carolin et al., J Hazard Mat. 407: 124827, 2021 (doi.org/10.1016/j.jhazmat.2020.124827); and Ref. 4: Arutchelvi et al., J Ind Microbiol Biotech. 35(12): 1559-1570, 2008 (doi.org/10.1007/s10295-008-0460-4).

Using provided fermentation and preparation methods, the biosurfactant yield obtained is higher than the level reported in the corresponding literature (citations above).

### Example 8

### Cost Estimate

This cost estimate includes comprehensive costs such as raw material cost, energy consumption, labor, and equipment loss. The cost of the biosurfactant produced by this method is compared with the currently reported or existing factory production situation.

TABLE 2
Cost estimate (USD/ton)

| | Raw | Energy | | Total |
|---|---|---|---|---|
| Source of cost | material | consumption | Purification | cost |
| Journal Literature | 4000-6000 | 2000-3000 | 8000-10000 | 14000-19000 |
| Existing factory | 3500-5500 | 2000-3000 | 4500-6500 | 10000-15000 |
| Pre-research foundation | <1000 | 1500-3000 | 1500-2500 | 4000-6500 |
| Method described herein | <200 | 500-1000 | 1000-2000 | 1700-3300 |
| Chemical surfactants | NA | NA | NA | 2000-4500 |

This estimation demonstrates that the methods described herein can effectively reduce the production cost of biosurfactants.

By the above comparative examples illustrate the preparation of the present disclosure biosurfactant green agent reasonable method, and synthetic surfactants was low cost, by different strains using metabolic differences may be achieved synthesized from carbon dioxide or lignocellulosic biological surfactant, the hair method is scientific, reasonable and effective.

The series of detailed descriptions provided above are only specific descriptions of representative feasible embodiments of the present disclosure. They are not intended to limit the scope of protection of the present disclosure, and any equivalent embodiments or embodiments made without departing from the technical spirit of the present disclosure. All changes shall be included in the protection scope of the present disclosure.

The amphoteric biological surface-active agents of the present disclosure, the characteristics, the biosurfactant of the present disclosure may be used in petroleum, food, cosmetic, environmental, pharmaceutical and other industries.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the disclosure provided herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present invention is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice.

What is claimed is:

1. A multi-stage, multi-strain fermentation method, comprising:
a first fermentation process, comprising contacting one or more strains of microorganism(s) with a primary feed stock comprising carbon dioxide, which first fermentation process produces a first fermentation broth;
a second fermentation process, comprising contacting one or more strains of microorganism(s) with a primary feed stock comprising lignocellulosic material, which second fermentation process produces a second fermentation broth; and
a third fermentation process, comprising contacting a mixture of the first fermentation broth and the second fermentation broth with one or more strains of microorganism under conditions and for a time sufficient to produce a biosurfactant fermentation broth, which biosurfactant fermentation broth comprises at least one biosurfactant compound.

2. The fermentation method of claim 1, wherein the at least one biosurfactant compound comprises a glycolipid or a lipopeptide.

3. The method of claim 1, which converts approximately one ton of sequestered CO₂ into 0.5 to 4.0 tons of biosurfactant.

4. The method of claim 1, wherein the one or more strains of microorganism(s) produce one or more lignocellulose decomposing enzymes, which enzymes convert lignocellulose into oligo-saccharide(s), monosaccharide(s), and/or small molecule acid species.

5. The method of claim 4, wherein the oligo-saccharide(s), monosaccharide(s), and/or small molecule acid species, and/or cells of the photo-autotrophic or chemo-autotrophic microorganism(s), are substrate(s) for the second fermentation process.

6. The method of claim 1, wherein the biosurfactant fermentation broth is further processed to extract biosurfactant.

7. The method of claim 1, further comprising a biosurfactant extraction process, the biosurfactant extraction process comprising:
fractionating the biosurfactant fermentation broth to produce an aqueous phase;
acid precipitating the aqueous liquid;
filtering the acid precipitated aqueous liquid to obtain the precipitate;
dissolving the precipitate with an aqueous liquid at pH>8 to produce a resuspended precipitate;
filtering the resuspended precipitate to obtain a aqueous solution containing the biosurfactant.

8. The method of claim 7, wherein the biosurfactant fermentation broth fractionation comprises adjusting the biosurfactant fermentation broth to pH=8.0-10.0 to produce a pH-adjusted broth, centrifuging the pH-adjusted broth at high speed (10000 ×g) to produce a microbial material pellet, a relatively clear aqueous liquid layer, and a lipid phase.

9. The method of claim 7, wherein the acid precipitation comprises adjusting the relatively clear aqueous liquid to pH=2-3 to produce an acidic aqueous liquid, then chilling the acidic aqueous liquid at 4-10° C. for 24 hours.

10. The method of claim 7, wherein filtering the acid precipitated aqueous liquid comprises filtering through a 0.1 pm ceramic membrane at 4-10° C.

11. The method of claim 7, wherein filtering the resuspended precipitate comprises filtering through a 0.1 μm ceramic membrane.

12. The method of claim 7, further comprising concentrating the biological surface-active aqueous solution under vacuum at 50° C.

13. The method of claim 1, wherein at least one microorganism of the first fermentation is selected from the following: Pyrococcus, Pseudomonas, Metallococcus sp., Metallosphaera, Rhodospirillum, Chloroflexus, Aspergillis, Cyanobacteria, Chlorella, Dunaliella, Nannochloropsis, Scenedesmus, and Botryococcus.

14. The method of claim 1, wherein at least one microorganism of the second fermentation is selected from the following: Aurantiacus, Sordaria, Pseudomonas, Trametes, Irpex, Lenzite, Phanerochaete, grams Klebsiella (Klebsiella), Ochrobactrum, Sphingobacterium, Dysgonomonas, Sphingobacterium, Bacteroides, Parabacteroides, Flavobacterium, polymorphonuclear Aeromonas, Pleomorphomonas, Arcticibacter, Elizabethkingia, Neisseria, Mycobacterium, Trichoderma (such as Trichoderma reesei), Zymomonas, Stenotrophomonas, Paenibacillus, Nocardia, Nocardiopsis, Nocardia, Bacillus, Rhizobium, Cellulomonas, Vibrio, Cellvibrio, Cytophaga, Alistipes, Aspergillus (such as Aspergillus flavus), Ruminofilibacter, and Clostridium.

15. The method of claim 1, wherein at least one microorganism of the third fermentation is selected from the following: Pseudomonas, Bacillus, Candida, Acinetobacter, Pantoea, Sphingomonas, Zymomonas, Streptomyces, Rhodococcus, Pseudozyma, Ustilaginales, and Moesziomyces.

16. The method claim 1, wherein the first fermentation process and the second fermentation process:
are carried out at least partially in separate containers;
are carried out at least partially in the same container; or
are substantially carried out in the same container (that is, mixed).

17. The method of claim 1, wherein the lignocellulose is from one or more of corn stalks, straws, leaves, and wood chips.

18. The method of claim 1, wherein the first fermentation process is carried out in a first culture condition comprising: culturing the microorganism(s) using mineral medium, carbon dioxide as the main carbon source, aeration of 0.01-0.4 vvm, and either light or no light conditions.

19. The method of claim 17, wherein the first culture condition further comprises an initial inoculum of 1-10% stock culture, temperature control 20-40° C., and agitation intensity 100-300 rpm.

20. The method of claim 17, wherein the first culture condition comprise a medium comprising:
potassium nitrate of 0.1-0.5%, potassium dihydrogen phosphate of 0.1-0.5%, magnesium sulphate heptahydrate of 0.01-0.2%, calcium chloride dihydrate of 0.01-0.05%, ferrous sulfate heptahydrate of 0.01-0.05%, zinc sulfate heptahydrate of 0.001-0.01%, manganese chloride tetrahydrate of 0.001-0.01%, copper sulfate pentahydrate of 0.001-0.01%, disodium edetate of 0.001-0.01%, and calcium carbonate of 0.1-0.3%.

21. The method of claim 1, wherein the first fermentation process is carried out for 4-10 days.

22. The method of claim 1, wherein the microorganism(s) of the second fermentation are cultured using mineral medium, with 2-10% lignocellulose as the main carbon source, maintained at 20-40° C., with stirring at 100-300 rpm, and an initial medium pH of 7-8.5.

23. The method of claim 22, wherein the culture medium volume was 50% (by volume); 10% inoculum (from seed stock).

24. The method of claim 22, wherein the medium comprises: (NH4)2SO4 0.1-0.5%, MgSO₄.7H₂O 0.01-0.05%, CuSO₄.5H₂O 0.01-0.03%, MnSO₄ 0.001-0.005%, and calcium carbonate 0.1-0.5%.

25. The method of claim 22, wherein the first and/or second fermentation process is carried out for 4-10 days.

26. The method of claim 1, wherein the third fermentation process is carried out using an inorganic salt medium, wherein the carbon source is a mixture of the first fermentation broth and the second fermentation broth.

27. The method of claim 26, wherein the mixture of the first and second fermentation broths is included in the third fermentation:
at a dosage of 5-15 wt %; or
at a dosage of 2-10 wt %.

28. The method of claim 26, wherein the temperature of the third fermentation is 20-40° C., the agitation intensity is 100-300 rpm, and ventilation is at a rate of 0.1-0.4 vvm.

29. The method of claim 26, wherein the third fermentation process is carried out for 3-10 days.

30. The method of claim 26, wherein the culture medium is at pH 6-7 and comprises: NaNO₃: 0.1-1.4%, FeCl₂: 0.002-0.006%, NaH₂PO₄: 0.25-1.5%, K₂HPO₄: 0.25-1.8%, MgSO_{4.}7H₂O: 0.005-0.015%, KCl: 0.05%-0.3%; Choline chloride: 0.05%-0.3%; yeast extract: 0.001-0.1%, and trace elements: Zn, Mn, Ca.

31. A biological surface-active agent (biosurfactant) produced using the method of claim 1.
